(19) United States
(12) Patent Application Publication
(10) Pub. No.: US 2008/0300235 A1
(43) Pub. Date: Dec. 4, 2008

(54) FXR Agonists for Reducing LOX-1 Expression

(75) Inventors: Douglas Harnish, Pennsburg, PA (US); Songwen Zhang, Lansdale, PA (US)

Correspondence Address: WYETH/FINNEGAN HENDERSON, LLP, 901 NEW YORK AVENUE, NW, WASHINGTON, DC, 20001-4413, US

(73) Assignee: Wyeth, Madison, NJ (US)

(21) Appl. No.: 12/130,322

(22) Filed: May 30, 2008

Related U.S. Application Data
(60) Provisional application No. 60/924,822, filed on Jun. 1, 2007.

(51) Int. Cl. A61K 31/55; C12N 5/00; A61P 9/00

(52) U.S. Cl. 514/213.01; 435/375

(57) ABSTRACT

Provided are certain methods of treating at least one disease state characterized by elevated expression of the Lectin-like Oxidized Low-density Lipoprotein Receptor 1 (LOX-1) in a patient with farnesoid X receptor agonists. Also provided are certain methods of reducing expression of LOX-1 in a cell with farnesoid X receptor agonists.

This application claims the benefit of priority to U.S. Provisional Application No. 60/924,822, filed Jun. 1, 2007, the entire contents of which are hereby incorporated herein by reference.

Provided are certain methods of treating at least one disease state characterized by elevated expression of Lectin-like Oxidized Low-density Lipoprotein Receptor 1 (LOX-1) in a patient with farnesoid X receptor agonists. Also provided are certain methods of modulating expression of LOX-1 in a cell, for example, reducing LOX-1 expression in a cell with farnesoid X receptor agonists. Also provided are methods of identifying a FXR modulator, methods of treating at least one disease state characterized by elevated expression of LOX-1 in a patient, methods of characterizing the risk that a patient will develop at least one disease state characterized by elevated expression of LOX-1, and methods of characterizing the level of FXR signaling in a mammal.

Nuclear receptors are a superfamily of regulatory proteins that are structurally and functionally related and are receptors for, e.g., steroids, retinoids, vitamin D and thyroid hormones (see, e.g., Evans (1988) Science 240:889-895). These proteins bind to cis-acting elements in the promoters of their target genes and modulate gene expression in response to ligands for the receptors.

Nuclear receptors can be classified based on their DNA binding properties (see, e.g., Evans, supra and Glass (1994) Endocr. Rev. 15:391-407). For example, one class of nuclear receptors includes the glucocorticoid, estrogen, androgen, progestin, and mineralocorticoid receptors which bind as homodimers to hormone response elements (HREs) organized as inverted repeats (see, e.g., Glass, supra). A second class of receptors, including those activated by retinoic acid, thyroid hormone, vitamin D₃, fatty acids/peroxisome proliferators (i.e., peroxisome proliferator activated receptor (PPAR)) and ecdysone, bind to HREs as heterodimers with a common partner, the retinoid X receptors (i.e., RXRs, also known as the 9-cis retinoic acid receptors; see, e.g., Levin et al. (1992) Nature 355:359-361 and Heyman et al. (1992) Cell 68:397-406).

RXRs are unique among the nuclear receptors in that they bind DNA as a homodimer and are required as a heterodimeric partner for a number of additional nuclear receptors to bind DNA (see, e.g., Mangelsdorf et al. (1995) Cell 83:841-850). The latter receptors, termed the class II nuclear receptor subfamily, include many which are established or implicated as important regulators of gene expression. There are three RXR genes (see, e.g., Mangelsdorf et al. (1992) Genes Dev. 6:329-344), coding for RXRα, -β, and -γ, all of which are able to heterodimerize with any of the class II receptors, although there appear to be preferences for distinct RXR subtypes by partner receptors in vivo (see, e.g., Chiba et al. (1997) Mol. Cell. Biol. 17:3013-3020). In the adult liver, RXRα is the most abundant of the three RXRs (see, e.g., Mangelsdorf et al. (1992) Genes Dev. 6:329-344), suggesting that it might have a prominent role in hepatic functions that involve regulation by class II nuclear receptors. See also, Wan et al. (2000) Mol. Cell. Biol 20:4436-4444.

The farnesoid X receptor (originally isolated as RIP14 (retinoid X receptor-interacting protein-14), see, e.g., Seol et al. (1995) Mol. Endocrinol. 9:72-85) is a member of the nuclear hormone receptor superfamily and is expressed in the liver, kidney, and intestine among other locations. It functions as a heterodimer with the retinoid X receptor (RXR) and binds to response elements in the promoters of target genes to regulate gene transcription. The farnesoid X receptor-RXR heterodimer binds with highest affinity to an inverted repeat-1 (IR-1) response element, in which consensus receptor-binding hexamers are separated by one nucleotide. The farnesoid X receptor is part of an interrelated process, in that the receptor is activated by bile acids (the end product of cholesterol metabolism) (see, e.g., Makishima et al. (1999) Science 284:1362-1365, Parks et al. (1999) Science 284:1365-1368, Wang et al. (1999) Mol. Cell. 3:543-553), which serve to inhibit cholesterol catabolism. See also, Urizar et al. (2000) J. Biol. Chem. 275:39313-39317.

Nuclear receptor activity, including the farnesoid X receptor has been implicated in a variety of diseases and disorders, including, but not limited to, hyperlipidemia and hypercholesterolemia, and complications thereof, including without limitation coronary artery disease, angina pectoris, carotid artery disease, strokes, cerebral arteriosclerosis, and xanthoma, (see, e.g., International Patent Application Publication No. WO 00/57915), hyperlipoproteinemia (see, e.g., International Patent Application Publication No. WO 01/60818), hypertriglyceridemia, lipodystrophy, peripheral occlusive disease, ischemic stroke, hyperglycemia, and diabetes mellitus (see, e.g., International Patent Application Publication No. WO 01/82917), disorders related to insulin resistance including the cluster of disease states, conditions or disorders that make up “Syndrome X” such as glucose intolerance, an increase in plasma triglyceride and a decrease in high-density lipoprotein cholesterol concentrations, hypertension, hyperuricemia, smaller denser low-density lipoprotein particles, and higher circulating levels of plasminogen activator inhibitor-1, atherosclerosis and gallstones (see, e.g., International Patent Application Publication No. WO 00/37077), disorders of the skin and mucous membranes (see, e.g., U.S. Pat. Nos. 6,184,215 and 6,187,814, and International Patent Application Publication No. WO 98/32444), obesity, acne (see, e.g., International Patent Application Publication No. WO 00/49992), and cancer, cholestasis, Parkinson's disease and Alzheimer's disease (see, e.g., International Patent Application Publication No. WO 00/17334).

Oxidative modification of low-density lipoprotein (LDL) is a key step in the pathogenesis of atherosclerosis. Oxidized LDL (ox-LDL), through a variety of scavenger receptors (SR), such as SR-AI/II, CD36, SR-BI, macrosialin/CD68 and SREC, is taken up by monocytes and macrophages and smooth muscle cells and exerts its pro-atherogenic effects on the vessel wall. The classic SRs are absent or present in very small amounts in endothelial cells. However, it has long been suggested that endothelial cells internalize and degrade the modified form of LDL, including ox-LDL, by cell-surface receptors.

Oxidized LDL leads to endothelial activation, dysfunction and injury. Endothelial activation is believed to be a very early step in the evolution of atherosclerosis. Activation of endothelial cells results in expression of a variety of genes, such as endothelin, tissue factor, cyclo-oxygenase, nitric oxide synthase (NOS), growth factors and monocyte chemoattractant protein-1 (MCP-1). It also leads to expression of adhesion molecules to which inflammatory cells attach, followed by a cascade of events, including cell rolling, separation of the intercellular junction and subendothelial migration of inflammatory cells. Oxidized LDL also induces apoptosis in endothelial cells.

Lectin-like Oxidized Low-density Lipoprotein Receptor 1 (LOX-1), also known as Oxidized Low-density Lipoprotein Receptor 1 (OLR-1), is a type II transmembrane receptor belonging to the class E scavenger receptor (SR-E) subfamily of the C-type lectin family. It binds and supports the internalization of multiple structurally unrelated macromolecules. It exists on the cell surface as covalent homodimers, which can further associate into non-covalently-linked oligomers. Cell surface LOX-1 can also be cleaved to release the soluble LOX-1 extracellular domain.

LOX-1 was identified as the major receptor for ox-LDL in endothelial cells. This receptor can support binding, internalization and proteolytic degradation of ox-LDL, but not significant amounts of acetylated LDL, which is a wellknown, high-affinity ligand for class A SRs expressed by endothelial cells (SR-EC).

LOX-1 is known to promote vascular inflammation and endothelial dysfunction and consequently is thought to play a pathogenic role in diseases such as heart failure, myocardial injury, diabetic nephropathy, hypertension, sepsis, osteoarthritis and rheumatoid arthritis; all indications not currently thought about in context of FXR. LOX-1 may also impact other disease processes, since LOX-1 binds other ligands including platelets, aged RBCs, apoptotic cells and advanced glycation end products. The expression of LOX-1 was initially described in endothelial cells (ECs), but has been demonstrated on numerous other cell types such as macrophages, smooth muscle cells and platelets.

LOX-1 activation has been shown to stimulate NF-κB and MAPK pathways, generate reactive oxygen species and inhibit nitric oxide production which all leads to endothelial dysfunction. LOX-1 is also cleaved at the membrane proximal extracellular domain and released from the cell surface. This soluble LOX-1 has been suggested to serve as a marker for early diagnosis of acute coronary syndromes. LOX-1 inhibition via blocking antibodies or antisense technology is associated with attenuation of sepsis, heart failure, rheumatoid arthritis, atherosclerosis and the associated ischemic injury. Therefore, LOX-1 may be a novel target for drug therapy.

Provided are methods of treating at least one disease state characterized by elevated expression of LOX-1 in a patient by administering to the patient a therapeutically effective amount of at least one farnesoid X receptor (FXR) agonist, where the at least one FXR agonist reduces expression of LOX-1 in the patient. In some embodiments the disease state is further characterized by at least one of endothelial dysfunction and vascular inflammation.

Also provided are methods of modulating expression of LOX-1 in a cell by administering an effective amount of at least one FXR modulator, to thereby modulate expression of LOX-1 in the cell. In some embodiments of the methods, a FXR agonist is administered to reduce LOX-1 expression in the cell.

Also provided are methods of identifying a FXR modulator by incubating a test agent with a cell; determining at least one of the following in the presence and/or absence of the test agent: (a) the expression of LOX-1 in the cell and (b) the secretion of soluble LOX-1 protein by the cell; and selecting a FXR modulator which fulfills at least one of the following features: (a) modulating expression of LOX-1 in the cell and (b) modulating secretion of soluble LOX-1 protein by the cell.

Also provided are methods of identifying a FXR modulator by providing a test agent to a cell; determining at least one of the following in the presence and/or absence of the test agent: (a) the level of NF-κB pathway signaling in the cell, (b) the level of MAPK pathway signaling in the cell, (c) production of reactive oxygen species by the cell, (d) nitric oxide production by the cell; and (e) production of at least one of soluble ICAM-1 and soluble VCAM-1 by the cell; and selecting a FXR modulator which fulfills at least one of the following features: (a) modulates the level of NF-κB pathway signaling in the cell, (b) modulates the level of MAPK pathway signaling in the cell, (c) modulates production of reactive oxygen species in the cell, (d) modulates nitric oxide production in the cell, and (e) modulates production of at least one of soluble ICAM-1 and soluble VCAM-1 by the cell.

Also provided are methods of treating at least one disease state characterized by elevated expression of LOX-1 in a patient by administering to a patient a therapeutically effective amount of at least one FXR agonist, wherein the at least one FXR agonist is identified by a method comprising: providing a test agent to a cell; determining at least one of the following in the presence and/or absence of the test agent: (a) the expression of LOX-1 in the cell and (b) the secretion of soluble LOX-1 protein by the cell; and selecting a FXR agonist which fulfills at least one of the following features: (a) reduces expression of LOX-1 in the cell and (b) reduces secretion of soluble LOX-1 protein by the cell.

Also provided are methods of treating at least one disease state characterized by elevated expression of LOX-1 in a patient by administering to a patient a therapeutically effective amount of at least one FXR agonist, wherein the at least one FXR agonist is identified by a method comprising: providing a test agent to a cell; determining at least one of the following in the presence and/or absence of the test agent: (a) the level of NF-κB pathway signaling in the cell, (b) the level of MAPK pathway signaling in the cell, (c) production of reactive oxygen species by the cell, (d) nitric oxide production by the cell; and (e) production of at least one of soluble ICAM-1 and soluble VCAM-1 by the cell; and selecting a FXR modulator which fulfills at least one of the following features: (a) modulates the level of NF-κB pathway signaling in the cell, (b) modulates the level of MAPK pathway signaling in the cell, (c) modulates production of reactive oxygen species in the cell, (d) modulates nitric oxide production in the cell, and (e) modulates production of at least one of soluble ICAM-1 and soluble VCAM-1 by the cell.

Also provided are methods of characterizing the risk that a patient will develop at least one disease state characterized by elevated expression of LOX-1 by measuring at least one of (a) the level of expression of a FXR gene in at least one tissue of the patient and (b) the level of FXR activity in at least one tissue of the patient.

Also provided are methods of characterizing the level of FXR signaling in a mammal by determining the level of circulating soluble LOX-1 protein in serum of the mammal and characterizing the level of FXR signaling in the mammal on the basis of the level of circulating soluble LOX-1 protein.

### DESCRIPTION OF DRAWINGS

FIG. 1 shows the effect of diet and an FXR agonist, Compound A (isopropyl 3-(3,4-difluorobenzoyl)-1,1-dimethyl-1,2,3,6-tetrahydroazepino[4,5-b]indole-5-carboxylate), on hepatic expression of LOX-1 in LDLR−/− mice.

FIG. 2 shows inhibition of LOX-1 target gene VCAM-1 by Compound A.

FIG. 3 shows the effect of Compound A on CD36 expression.

FIG. 4A and FIG. 4B show regulation of LOX-1 by FXR in the diabetic mouse strain KKAy.

FIG. 5A and FIG. 5B show regulation of serum sLOX-1 by FXR in the diabetic mouse strain KKAy and various non-diabetic mouse strains.

FIG. 6 shows that inhibition of LOX-1 and VCAM-1 gene expression by Compound A is dependent upon FXR.

As used in the present specification, the following words and phrases are generally intended to have the meanings as set forth below, except to the extent that the context in which they are used indicates otherwise.

As used herein, the terms “treat”, “treating”, and “treatment” refer to any manner in which one or more of the symptoms of a disease or disorder are beneficially altered so as to prevent or delay the onset, retard the progression, or ameliorate the symptoms of a disease or disorder.

As used herein the phrase “therapeutically effective amount” refers to the amount sufficient to provide a therapeutic outcome regarding at least one symptom of a disease or condition.

As used herein, the term “farnesoid X receptor (FXR)” refers to all mammalian forms of such receptor including, for example, alternative splice isoforms and naturally occurring isoforms (see, e.g. Huber et al, Gene (2002), Vol. 290, pp.: 35-43). Representative farnesoid X receptor species include, without limitation the rat (GenBank Accession No. NM_{—}021745), mouse (Genbank Accession No. NM_{—}009108), and human (GenBank Accession No. NM_{—}005123) forms of the receptor.

As used herein “Lectin-like Oxidized Low-density Lipoprotein Receptor 1 (LOX-1)”, also known as Oxidized Low-density Lipoprotein Receptor 1 (OLR-1), refers to all mammalian forms of such receptor including, for example, alternative splice isoforms and naturally occurring isoforms. Representative LOX-1 species include, without limitation the human (GenBank Accession No. NM_{—}002543), mouse (GenBank Accession No. NM_{—}138648) and rat (GenBank Accession No. NM_{—}133306) forms of the receptor.

As used herein, a reference to “expression” of LOX-1 refers to expression of LOX-1 mRNA and/or LOX-1 protein, except to the extent that the context indicates that one or the other is exclusively intended. In some embodiments expression of LOX-1 mRNA is used. In some embodiments expression of LOX-1 protein in used, which is some embodiments can be expression of soluble LOX-1 protein. In some embodiments expression of LOX-1 mRNA and expression of LOX-1 protein, which can be expression of soluble LOX-1 protein, are both used. LOX-1 expression may be measured, for example, by measuring LOX-1 mRNA expression levels or by measuring LOX-1 protein levels, including for example by measuring the level of serum soluble LOX-1.

Endothelium is the layer of thin specialized epithelium, comprising a simple squamous layer of cells that line the interior surface of blood vessels, forming an interface between circulating blood in the lumen and the rest of the vessel wall (simple squamous epithelium). Endothelial cells line the entire circulatory system, from the heart to the smallest capillary. As used herein, the term “endothelial dysfunction” refers to a physiological dysfunction of at least one normal process carried out by the endothelium. Normal functions of endothelial cells include, by way of example only, mediation of coagulation, platelet adhesion, immune function, control of volume and electrolyte content of the intravascular and extravascular spaces. Compromise of normal function of endothelial cells is characteristic of endothelial dysfunction. Endothelial dysfunction can result, for example, from disease processes, as occurs in septic shock, hypertension, hypercholesterolaemia, and diabetes, as well as from environmental factors, such as from smoking tobacco products. This dysfunction includes, for example, at least one of a reduction in nitric oxide production, induction of inflammatory signaling cascades such as NF-κB and MAPK, production of reactive oxygen species and endothelial apoptosis.

As used herein, the term “vascular inflammation” refers to the resulting pathology induced by endothelial dysfunction, and can include, for example, at least one of induction of scavenger receptors, induction of adehesion molecules, and chemokine and cytokine expression resulting in the recruitment of LDL, oxLDL as well as mononcytes and macrophages to the endothelium.

As used herein, the term “heart failure” refers to any structural or functional cardiac disorder that impairs the ability of the heart to fill with or pump a sufficient amount of blood throughout the body. Heart failure can be classified, for example, by the side of the heart involved (left heart failure versus right heart failure), whether the abnormality is due to contraction or relaxation of the heart (systolic heart failure vs. diastolic heart failure), and whether the abnormality is due to low cardiac output or low systemic vascular resistance (low-output heart failure vs. high-output heart failure).

As used herein, the term “myocardial injury” refers to a contusion or bruising of the myocardium, such as from blunt trauma, as well as to ischaemic injury to the myocardium, such as results from angina (including unstable angina) or myocardial infarction, for example.

As used herein, the term “dimethylarginine dimethylaminohydrolase 1 (DDAH1)” refers to all mammalian forms including, for example, alternative splice isoforms and naturally occurring isoforms. Representative DDAH1 species include, without limitation the human (GenBank Accession No. NM_{—}012137), mouse (GenBank Accession No. NM_{—}026993) and rat (GenBank Accession No. NM_{—}022297) forms.

As used herein, the term “argininosuccinate synthetase 1 (ASS1)” refers to all mammalian forms including, for example, alternative splice isoforms and naturally occurring isoforms. Representative ASS1 species include, without limitation the human transcript variant 1 (GenBank Accession No. NM_{—}000050), human transcript variant 2 (GenBank Accession No. NM_{—}054012), mouse (GenBank Accession No. NM_{—}007494) and rat (GenBank Accession No. NM_{—}013157) forms.

As used herein, the term “GTP cyclohydrolase 1 (GTPCH)” refers to all mammalian forms of the protein including, for example, alternative splice isoforms and naturally occurring isoforms. Representative GTPCH species include, without limitation the human transcript variant 4 (GenBank Accession No. NM_{—}001024071), human transcript variant 3 (GenBank Accession No. NM_{—}001024070), and mouse (GenBank Accession No. NM_{—}008102) forms.

As used herein, the phrase “NF-κB pathway signaling” refers to any signaling pathway that comprises NF-κB. In some embodiments, NF-κB pathway signaling is measured by measuring the activity or state of NF-κB. In some embodiments NF-κB pathway signaling is measured by measuring the activity or state of a molecule downstream of NF-κB in a signaling pathway. In certain embodiments NF-κB pathway signaling is measured by measuring the activity or state of a molecule upstream of NF-κB in a signaling pathway.

As used herein, the phrase “MAPK pathway signaling” refers to any signaling pathway that comprises MAPK. In some embodiments, MAPK pathway signaling is measured by measuring the activity or state of MAPK. In some embodiments MAPK pathway signaling is measured by measuring the activity or state of a molecule downstream of MAPK in a signaling pathway. In some embodiments MAPK pathway signaling is measured by measuring the activity or state of a molecule upstream of MAPK in a signaling pathway.

As used herein, the term “atherosclerosis” refers to a condition in which patchy deposits of fatty material (atheromas or atherosclerotic plaques) develop in the walls of medium-sized and large arteries, leading to reduced or blocked blood flow.

As used herein, the term “diabetic nephropathy” (also known as Kimmelstiel-Wilson syndrome and intercapillary glomerulonephritis), is a progressive kidney disease caused by angiopathy of capillaries in the kidney glomeruli. It is characterized by nephrotic syndrome and nodular glomerulosclerosis, caused by longstanding diabetes mellitus.

As used herein, the term “hypertension” refers to a medical condition in which a patients blood pressure is chronically elevated. In embodiments the patients blood pressure is above about 140/90.

As used herein, the term “sepsis” refers to a systemic inflammatory response causing widespread activation of inflammation and coagulation pathways. Sepsis is considered present if infection is highly suspected or proven and at least two of the following systemic inflammatory response syndrome (SIRS) criteria are met:

Heart rate>90 beats per minute;

Body temperature<36° C. (96.8° F.) or >38° C. (100.4° F.);

Hyperventilation (high respiratory rate)>20 breaths per minute or, on blood gas, a PaCO₂ less than 32 mm Hg; and

White blood cell count<4000 cells/mm³ or >12000 cells/mm³ (<4×10⁹ or >12×10⁹ cells/L), or greater than 10% band forms (immature white blood cells).

As used herein, the term “osteoarthritis” refers to a condition in which low-grade inflammation results in pain in the joints, caused by wearing of the cartilage that covers and acts as a cushion inside joints. Osteoarthritis commonly affects the hands, feet, spine, and the large weight-bearing joints, such as the hips and knees, although in theory, any joint in the body can be affected. As osteoarthritis progresses, the affected joints appear larger, are stiff and painful, and usually feel worse, the more they are used throughout the day, thus distinguishing it from rheumatoid arthritis.

As used herein, the term “rheumatoid arthritis” refers to a chronic, inflammatory, multisystem, autoimmune disorder. It is commonly polyarticular, i.e. it affects many joints. The symptoms that distinguish rheumatoid arthritis from other forms of arthritis are inflammation and soft-tissue swelling of many joints at the same time (polyarthritis). The joints are usually affected initially asymmetrically and then in a symmetrical fashion as the disease progresses. The pain generally improves with use of the affected joints, and there is usually stiffness of all joints in the morning that lasts over 1 hour. Thus, the pain of rheumatoid arthritis is usually worse in the morning compared to the classic pain of osteoarthritis where the pain worsens over the day as the joints are used.

As used herein, “Monocyte chemotactic protein-1 (MCP-1)”, also known as chemokine (C-C motif) ligand 2 (CCL2), refers to all mammalian forms of the protein. Representative MCP-1 species include, without limitation, the human (GenBank Accession No. NM_{—}002982), mouse (GenBank Accession No. NM_{—}011333) and rat (GenBank Accession No NM_{—}031530) forms.

As used herein, “VCAM-1” refers to all mammalian forms of the protein including, for example, alternative splice isoforms and naturally occurring isoforms. Representative VCAM-1 species include, without limitation the human variant 1 (GenBank Accession No. NM_{—}001078), human variant 2 (GenBank Accession No. NM_{—}080682), mouse (GenBank Accession No. NM_{—}011693) and rat (GenBank Accession No. NM_{—}012889) forms.

As used herein, “ICAM-1” refers to all mammalian forms of the protein including, for example, alternative splice isoforms and naturally occurring isoforms. Representative ICAM-1 species include, without limitation the human (GenBank Accession No. NM_{—}000201), mouse (GenBank Accession No. NM_{—}010493) and rat (GenBank Accession No. NM_{—}012967) forms.

As used herein, the term “agonist” refers to an agent that triggers a response that is at least one response triggered by binding of an endogenous ligand of the receptor to the receptor. In some embodiments, the agonist may act directly or indirectly on a second agent that itself modulates the activity of the receptor. In some embodiments, the at least one response of the receptor is an activity of the receptor that can be measured with assays including but not limited to physiological, pharmacological, and biochemical assays. Exemplary assays include but are not limited to assays that measure the binding of an agent to the receptor, the binding of the receptor to a substrate such as but not limited to a nuclear receptor and a regulatory element of a target gene, the effect on gene expression assayed at the mRNA or resultant protein level, and the effect on an activity of proteins regulated either directly or indirectly by the receptor. For example, farnesoid X receptor activity may be measured by monitoring expression of LOX-1.

As used herein, the term “agent” or “active agent” refers to a substance including, but not limited to a chemical compound, such as a small molecule or a complex organic compound, a protein, such as an antibody or antibody fragment or a protein comprising an antibody fragment, or a genetic construct which acts at the DNA or mRNA level in an organism.

As used herein, the term “expression” of a polynucleotide or gene refers to the production of a RNA transcript. Because an RNA transcript encoded by a gene is translated into a protein the level of expression of a gene may be measured by directly assaying the level of mRNA produced or indirectly by assaying the level of protein produced.

As used herein, the term “coadministering” refers to a dosage regimen for a first agent that overlaps with the dosage regimen of a second agent, or to simultaneous administration of the first agent and the second agent. A dosage regimen is characterized by dosage amount, frequency, and duration. Two dosage regimens overlap if between a first and a second administration of a first agent the second agent is administered.

As used herein, the phrase “effective amount” refers to the amount sufficient to increase or reduce a specified activity, function, or feature.

As used herein, the term “modulating” and “modulate” refers to changing or altering an activity, function, or feature. The term “modulator” refers to an agent which modulates an activity, function, or feature. For example, an agent may modulate an activity by increasing or decreasing the activity compared to the effects on the activity in the absence of the agent. In some embodiments, a modulator that increases an activity, function, or feature is an agonist.

Provided is a method of treating at least one disease state characterized by elevated expression of the Lectin-like Oxidized Low-density Lipoprotein Receptor 1 (LOX-1) in a patient by administering to the patient a therapeutically effective amount of at least one farnesoid X receptor (FXR) agonist, where the at least one FXR agonist reduces expression of LOX-1 in the patient. In some embodiments the disease state is further characterized by at least one of endothelial dysfunction and vascular inflammation. In some embodiments the at least one disease state is selected from heart failure, myocardial injury, atherosclerosis, diabetic nephropathy, hypertension, sepsis, osteoarthritis, and rheumatoid arthritis. In some embodiments the heart failure comprises at least one of left sided heart failure, right sided heart failure, systolic heart failure, and diastolic heart failure. In some embodiments the myocardial injury comprises at least one of unstable angina and myocardial infarction. In some embodiments the FXR agonist reduces at least one of NF-κB pathway signaling, MAPK pathway signaling, and production of reactive oxygen species in the patient. In some embodiments the FXR agonist increases nitric oxide production in the patient. In some embodiments LOX-1 expression is reduced in at least one tissue of the patient selected from heart, liver, and kidney. In some embodiments LOX-1 expression is reduced in at least one cell type of the patient selected from endothelial cells, macrophages, smooth muscle cells, dendritic cells, cardiac myocytes, and platelets. In some embodiments the level of serum soluble LOX-1 protein in the patient is reduced. In some embodiments expression of at least one LOX-1 target selected from MCP-1, VCAM-1, and ICAM-1 is reduced in the patient. In some embodiments expression of at least one FXR target selected from DDAH1, ASS1, and GTPCH is increased in the patient. In some embodiments the level of assymetric dimethylarginine (ADMA) is reduced in the patient. In some embodiments expression of nitric oxide synthase is increased in the patient. In some embodiments the LOX-1 expression level in the patient is reduced to about the level of LOX-1 expression in the absence of the disease state. In some embodiments the LOX-1 expression level in the patient is reduced to below about a threshold level of LOX-1 expression. In some embodiments the threshold level of LOX-1 expression is higher than the level of LOX-1 expression in the absence of the disease state.

Also provided is a method of modulating expression of LOX-1 in a cell by administering an effective amount of at least one FXR modulator, to thereby modulate expression of LOX-1 in the cell. In some embodiments LOX-1 expression is reduced and the at least one FXR modulator is a FXR agonist. In some embodiments the FXR agonist reduces at least one of NF-κB pathway signaling, MAPK pathway signaling, and production of reactive oxygen species by the cell. In some embodiments the FXR agonist increases nitric oxide production by the cell. In some embodiments expression of at least one LOX-1 target selected from MCP-1, VCAM-1 and ICAM-1 is reduced in the cell. In some embodiments expression of at least one FXR target selected from DDAH1, ASS1, and GTPCH is increased in the cell. In some embodiments the level of ADMA is reduced in the patient. In some embodiments expression of nitric oxide synthase is increased in the cell.

Also provided is a method of identifying a FXR modulator by incubating a test agent with a cell; determining at least one of the following in the presence and/or absence of the test agent: (a) the expression of LOX-1 in the cell and (b) the secretion of soluble LOX-1 protein by the cell; and selecting a FXR modulator which fulfills at least one of the following features: (a) modulating expression of LOX-1 in the cell and (b) modulating secretion of soluble LOX-1 protein by the cell. In some embodiments the FXR modulator is a FXR agonist and the FXR agonist fulfills at least one of the following features: (a) reducing expression of LOX-1 in the cell and (b) reducing secretion of soluble LOX-1 protein by the cell.

Also provided is a method of identifying a FXR modulator by providing a test agent to a cell; determining at least one of the following in the presence and/or absence of the test agent: (a) the level of NF-κB pathway signaling in the cell, (b) the level of MAPK pathway signaling in the cell, (c) production of reactive oxygen species by the cell, (d) nitric oxide production by the cell; and (e) production of at least one of soluble ICAM-1 and soluble VCAM-1 by the cell; and selecting a FXR modulator which fulfills at least one of the following features: (a) modulates the level of NF-κB pathway signaling in the cell, (b) modulates the level of MAPK pathway signaling in the cell, (c) modulates production of reactive oxygen species in the cell, (d) modulates nitric oxide production in the cell, and (e) modulates production of at least one of soluble ICAM-1 and soluble VCAM-1 by the cell. In some embodiments the FXR modulator is a FXR agonist and the FXR agonist fulfills at least one of the following features: (a) reduces the level of NF-κB pathway signaling in the cell, (b) reduces the level of MAPK pathway signaling in the cell, (c) reduces production of reactive oxygen species in the cell, (d) increases nitric oxide production in the cell, and (e) reduces production of at least one of soluble ICAM-1 and soluble VCAM-1 by the cell.

Also provided is a method of treating at least one disease state characterized by elevated expression of LOX-1 in a patient by administering to a patient a therapeutically effective amount of at least one FXR agonist, wherein the at least one FXR agonist is identified by a method comprising: providing a test agent to a cell; determining at least one of the following in the presence and/or absence of the test agent: (a) the expression of LOX-1 in the cell and (b) the secretion of soluble LOX-1 by the cell; and selecting a FXR agonist which fulfills at least one of the following features: (a) reduces expression of LOX-1 in the cell and (b) reduces secretion of soluble LOX-1 protein by the cell.

Also provided is a method of treating at least one disease state characterized by elevated expression of LOX-1 in a patient by administering to a patient a therapeutically effective amount of at least one FXR agonist, wherein the at least one FXR agonist is identified by a method comprising: providing a test agent to a cell; determining at least one of the following in the presence and/or absence of the test agent: (a) the level of NF-κB pathway signaling in the cell, (b) the level of MAPK pathway signaling in the cell, (c) production of reactive oxygen species by the cell, (d) nitric oxide production by the cell, and (e) production of at least one of soluble ICAM-1 and soluble VCAM-1 by the cell; and selecting a FXR agonist which fulfills at least one of the following features: (a) reduces the level of NF-κB pathway signaling in the cell, (b) reduces the level of MAPK pathway signaling in the cell, (c) reduces production of reactive oxygen species in the cell, (d) increases nitric oxide production in the cell, and (e) reduces production of at least one of soluble ICAM-1 and soluble VCAM-1 by the cell.

Also provided is a method of characterizing the risk that a patient will develop at least one disease state characterized by elevated expression of LOX-1 by measuring at least one of (a) the level of expression of a FXR gene in at least one tissue of the patient and (b) the level of FXR activity in at least one tissue of the patient.

Also provided is a method of characterizing the level of FXR signaling in a mammal by determining the level of circulating soluble LOX-1 protein in serum of the mammal and characterizing the level of FXR signaling in the mammal on the basis of the level of circulating soluble LOX-1 protein. In some embodiments the level of circulating soluble LOX-1 protein is above about a predetermined threshold and the level of FXR signaling is determined to be below about a predetermined threshold. In some embodiments the level of circulating soluble LOX-1 protein is below about a predetermined threshold and the level of FXR signaling is determined to be above about a predetermined threshold. In some embodiments the level of FXR signaling in the mammal is determined to be characteristic of a disease state. In some embodiments the level of FXR signaling in the mammal is determined to be therapeutic. In some embodiments the mammal is a human.

In some embodiments of the methods provided herein the FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl) 1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoyl)-1,4,4-trimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro-4H-thieno[2,3-D]azepine-8-carboxylic acid ethyl ester;
6-(4-fluoro-benzoyl)-3,6,7,8-tetrahydro-imidazo[4,5-D]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide;
diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate;
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3(4-fluorobenzoyl)-2-methyl-1,2,3,6-tetrahydroazepino[4,5-b]indole-5-carboxylate; and
n-propyl 3(4-fluorobenzoyl)-2-methyl-8-fluoro-1,2,3,6-tetrahydroazepino[4,5-b]indole-5-carboxylate.

In some embodiments of the methods the FXR modulator or agonist is selected from a compound disclosed in U.S. Patent Application Publication No. 2004/0023947A1, published Feb. 5, 2004, U.S. Patent Application Publication No. 2005/0054634A1, published Mar. 10, 2005, and U.S. Patent Application Publication No. 2007/0015746A1, published Jan. 18, 2007, each of which are hereby incorporated herein by reference.

Pharmaceutical compositions for use in the methods herein are formulated to contain therapeutically effective amounts of at least one farnesoid X receptor modulator. The pharmaceutical compositions are useful, for example, in the treatment of at least one disease state characterized by elevated expression of LOX-1.

In some embodiments, the at least one farnesoid X receptor modulator is formulated into a suitable pharmaceutical preparation such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the farnesoid X modulator described above is formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more farnesoid X modulators or pharmaceutically acceptable derivatives is (are) mixed with a suitable pharmaceutical carrier or vehicle.

Pharmaceutically acceptable derivatives include acids, bases, enol ethers and esters, salts, esters, hydrates, solvates and prodrug forms. The derivative is selected such that its pharmacokinetic properties are superior with respect to at least one characteristic to the corresponding neutral agent. The farnesoid X modulator may be derivatized prior to formulation.

The concentrations of the farnesoid X modulator in the compositions are effective for delivery of an amount, upon administration, that treats one or more of the symptoms of at least one disease state characterized by elevated expression of LOX-1.

Typically, by way of example and without limitation, the compositions are formulated for single dosage administration. To formulate a composition, the weight fraction of farnesoid X modulator is dissolved, suspended, dispersed or otherwise mixed in a selected vehicle at an effective concentration such that the treated condition, a disease state characterized by elevated expression of LOX-1, is relieved or ameliorated. Pharmaceutical carriers or vehicles suitable for administration of the farnesoid X modulator include any such carriers known to those skilled in the art to be suitable for the particular mode of administration.

In addition, the farnesoid X modulator may be formulated as the sole active agent in the composition or may be combined with other active agents. Liposomal suspensions, including tissue-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a farnesoid X modulator provided herein in phosphate buffered saline lacking divalent cations (PBS) is added and the flask shaken until the lipid film is dispersed. The resulting vesicles are washed to remove unencapsulated farnesoid X modulator, pelleted by centrifugation, and then resuspended in PBS.

The active farnesoid X modulator is included in the pharmaceutically acceptable carrier in an amount sufficient to exert a therapeutically useful effect in the absence of undesirable side effects on the patient treated. The therapeutically effective concentration may be determined empirically by testing the agents in in vitro and in vivo systems described herein and in International Patent Application Publication Nos. 99/27365 and 00/25134 and then extrapolated there from for dosages for humans.

The concentration of active farnesoid X modulator in the pharmaceutical composition will depend on absorption, inactivation and excretion rates of the active agent, the physicochemical characteristics of the agent, the dosage schedule, and amount administered as well as other factors known to those of skill in the art. For example, the amount that is delivered is sufficient to treat at least one disease state characterized by elevated expression of LOX-1 as described herein.

Typically a therapeutically effective dosage should produce a serum concentration of active agent of from about 0.1 ng/ml to about 50-100 μg/ml. The pharmaceutical compositions typically should provide a dosage of from about 0.001 mg to about 2000 mg of farnesoid X modulator per kilogram of body weight per day. Pharmaceutical dosage unit forms are prepared to provide from about 1 mg to about 1000 mg, such as from about 10 to about 500 mg of the active agent or a combination of agents per dosage unit form.

The active agent may be administered at once, or may be divided into a number of smaller doses to be administered at intervals of time. It is understood that the precise dosage and duration of treatment is a function of the disease state characterized by elevated expression of LOX-1 being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed methods.

Thus, effective concentrations or amounts of one or more farnesoid X modulators or pharmaceutically acceptable derivatives thereof are mixed with a suitable pharmaceutical carrier or vehicle for systemic, topical or local administration to form pharmaceutical compositions. Farnesoid X modulators are included in an amount effective for treating at least one disease state characterized by elevated expression of LOX-1. The concentration of active agent in the composition will depend on absorption, inactivation, excretion rates of the active agent, the dosage schedule, amount administered, particular formulation as well as other factors known to those of skill in the art.

The compositions are intended to be administered by a suitable route, including by way of example and without limitation orally, parenterally, rectally, topically and locally. For oral administration, capsules and tablets can be used. The compositions are in liquid, semi-liquid or solid form and are formulated in a manner suitable for each route of administration.

Solutions or suspensions used for parenteral, intradermal, subcutaneous, or topical application can include any of the following components, in any combination: a sterile diluent, including by way of example without limitation, water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampoules, disposable syringes or single or multiple dose vials made of glass, plastic or other suitable material.

In instances in which the agents exhibit insufficient solubility, methods for solubilizing agents may be used. Such methods are known to those of skill in this art, and include, but are not limited to, using co-solvents, such as dimethylsulfoxide (DMSO), using surfactants, such as TWEEN®, or dissolution in aqueous sodium bicarbonate. Pharmaceutically acceptable derivatives of the agents may also be used in formulating effective pharmaceutical compositions.

Upon mixing or addition of the agent(s), the resulting mixture may be a solution, suspension, emulsion or the like. The form of the resulting mixture depends upon a number of factors, including the intended mode of administration and the solubility of the agent in the selected carrier or vehicle. The effective concentration is sufficient for treating one or more symptoms of at least one disease state characterized by elevated expression of LOX-1 and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the agents or pharmaceutically acceptable derivatives thereof. The pharmaceutically therapeutically active agents and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active agent sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active agent, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active agent as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active agent in an amount sufficient to alleviate the symptoms of the treated subject.

Dosage forms or compositions containing active agent in the range of 0.005% to 100% with the balance made up from non-toxic carrier may be prepared. For oral administration, a pharmaceutically acceptable non-toxic composition is formed by the incorporation of any of the normally employed excipients, such as, for example and without limitation, pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% active agent, such as 0.1-85%, or such as 75-95%.

The active agents or pharmaceutically acceptable derivatives may be prepared with carriers that protect the agent against rapid elimination from the body, such as time release formulations or coatings. The compositions may include other active agents to obtain desired combinations of properties. FXR modulators or pharmaceutically acceptable derivatives thereof, may also be advantageously administered for therapeutic or prophylactic purposes together with another pharmacological agent known in the general art to be of value in treating at least one disease state characterized by elevated expression of LOX-1.

Oral pharmaceutical dosage forms include, by way of example and without limitation, solid, gel and liquid. Solid dosage forms include tablets, capsules, granules, and bulk powders. Oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In some embodiments, the formulations are solid dosage forms, such as capsules or tablets. The tablets, pills, capsules, troches and the like can contain any of the following ingredients, or agents of a similar nature: a binder; a diluent; a disintegrating agent; a lubricant; a glidant; a sweetening agent; and a flavoring agent.

Examples of binders include, by way of example and without limitation, microcrystalline cellulose, gum tragacanth, glucose solution, acacia mucilage, gelatin solution, sucrose, and starch paste. Lubricants include, by way of example and without limitation, talc, starch, magnesium or calcium stearate, lycopodium and stearic acid. Diluents include, by way of example and without limitation, lactose, sucrose, starch, kaolin, salt, mannitol, and dicalcium phosphate. Glidants include, by way of example and without limitation, colloidal silicon dioxide. Disintegrating agents include, by way of example and without limitation, crosscarmellose sodium, sodium starch glycolate, alginic acid, corn starch, potato starch, bentonite, methylcellulose, agar and carboxymethylcellulose. Coloring agents include, by way of example and without limitation, any of the approved certified water soluble FD and C dyes, mixtures thereof; and water insoluble FD and C dyes suspended on alumina hydrate. Sweetening agents include, by way of example and without limitation, sucrose, lactose, mannitol and artificial sweetening agents such as saccharin, and any number of spray dried flavors. Flavoring agents include, by way of example and without limitation, natural flavors extracted from plants such as fruits and synthetic blends of agents which produce a pleasant sensation, such as, but not limited to peppermint and methyl salicylate. Wetting agents include, by way of example and without limitation, propylene glycol monostearate, sorbitan monooleate, diethylene glycol monolaurate, and polyoxyethylene laural ether. Emetic-coatings include, by way of example and without limitation, fatty acids, fats, waxes, shellac, ammoniated shellac and cellulose acetate phthalates. Film coatings include, by way of example and without limitation, hydroxyethylcellulose, sodium carboxymethylcellulose, polyethylene glycol 4000 and cellulose acetate phthalate.

If oral administration is desired, the agent could be provided in a composition that protects it from the acidic environment of the stomach. For example, the composition can be formulated in an enteric coating that maintains its integrity in the stomach and releases the active agent in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The agents can also be administered as a component of an elixir, suspension, syrup, wafer, sprinkle, chewing gum or the like. A syrup may contain, in addition to the active agents, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents may be used in any of the above dosage forms.

Solvents, include by way of example and without limitation, glycerin, sorbitol, ethyl alcohol and syrup. Examples of preservatives include without limitation glycerin, methyl and propylparaben, benzoic add, sodium benzoate and alcohol. Non-aqueous liquids utilized in emulsions, include by way of example and without limitation, mineral oil and cottonseed oil. Emulsifying agents, include by way of example and without limitation, gelatin, acacia, tragacanth, bentonite, and surfactants such as polyoxyethylene sorbitan monooleate. Suspending agents include, by way of example and without limitation, sodium carboxymethylcellulose, pectin, tragacanth, Veegum and acacia. Diluents include, by way of example and without limitation, lactose and sucrose. Sweetening agents include, by way of example and without limitation, sucrose, syrups, glycerin and artificial sweetening agents such as saccharin. Wetting agents, include by way of example and without limitation, propylene glycol monostearate, sorbitan monooleate, diethylene glycol monolaurate, and polyoxyethylene lauryl ether. Organic acids include, by way of example and without limitation, citric and tartaric acid. Sources of carbon dioxide include, by way of example and without limitation, sodium bicarbonate and sodium carbonate. Coloring agents include, by way of example and without limitation, any of the approved certified water soluble FD and C dyes, and mixtures thereof. Flavoring agents include, by way of example and without limitation, natural flavors extracted from plants such fruits, and synthetic blends of agents which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active agent or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a agent provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

Tablets and capsules formulations may be coated as known by those of skill in the art in order to modify or sustain dissolution of the active ingredient. Thus, for example and without limitation, they may be coated with a conventional enterically digestible coating, such as phenylsalicylate, waxes and cellulose acetate phthalate.

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients, include by way of example and without limitation, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a FXR modulator is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The agent diffuses through the outer polymeric membrane in a release rate controlling step. The percentage of active agent contained in such parenteral compositions is highly dependent on the specific nature thereof, as well as the activity of the agent and the needs of the subject.

Parenteral administration of the FXR modulators includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Aqueous vehicles include, by way of example and without limitation, Sodium Chloride Injection, Ringers Injection, Isotonic Dextrose Injection, Sterile Water Injection, Dextrose and Lactated Ringers Injection. Nonaqueous parenteral vehicles include, by way of example and without limitation, fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include, by way of example and without limitation, sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include, by way of example and without limitation, ethyl alcohol, polyethylene glycol and propylene glycol for water miscible vehicles and sodium hydroxide, hydrochloric acid, citric acid or lactic acid for pH adjustment.

The concentration of the pharmaceutically active agent is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. Preparations for parenteral administration should be sterile, as is known and practiced in the art.

Illustratively, intravenous or intraarterial infusion of a sterile aqueous solution containing an active agent is an effective mode of administration. Another embodiment is a sterile aqueous or oily solution or suspension containing an active agent injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active agent to the treated tissue(s). The active agent may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The agent may be suspended in micronized or other suitable form or may be derivatized, e.g., to produce a more soluble active product or to produce a prodrug or other pharmaceutically acceptable derivative. The form of the resulting mixture depends upon a number of factors, including the intended mode of administration and the solubility of the agent in the selected carrier or vehicle. The effective concentration is sufficient for ameliorating the symptoms of the condition and may be empirically determined.

Lyophilized powders can be reconstituted for administration as solutions, emulsions, and other mixtures or formulated as solids or gels.

The sterile, lyophilized powder is prepared by dissolving a agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain, by way of example and without limitation, a single dosage (10-1000 mg, such as 100-500 mg) or multiple dosages of the agent. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, such as about 5-35 mg, for example, about 9-30 mg of lyophilized powder, is added per mL of sterile water or other suitable carrier. The precise amount depends upon the selected agent. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The agents or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, by way of example and without limitation, have diameters of less than about 50 microns, such as less than about 10 microns.

The agents may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active agent alone or in combination with other pharmaceutically acceptable excipients can also be administered.

These solutions, particularly those intended for ophthalmic use, may be formulated, by way of example and without limitation, as about 0.01% to about 10% isotonic solutions, pH about 5-7, with appropriate salts.

Other routes of administration, such as transdermal patches, and rectal administration are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

Pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is, by way of example and without limitation, about 2 to 3 gm.

Tablets and capsules for rectal administration are manufactured using the same pharmaceutically acceptable substance and by the same methods as for formulations for oral administration.

The FXR modulators, or pharmaceutically acceptable derivatives thereof, may also be formulated to be targeted to a particular tissue, receptor, or other area of the body of the subject to be treated. Many such targeting methods are well known to those of skill in the art. Such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a agent provided herein in phosphate buffered saline lacking divalent cations (PBS) is added and the flask shaken until the lipid film is dispersed. The resulting vesicles are washed to remove unencapsulated agent, pelleted by centrifugation, and then resuspended in PBS.

The FXR modulators or pharmaceutically acceptable derivatives for use in the methods may be packaged as articles of manufacture containing packaging material, a FXR modulator or pharmaceutically acceptable derivative thereof provided herein, which is effective for modulating the activity of a farnesoid X receptor or for treatment, of one or more symptoms of at least one disease state characterized by elevated expression of LOX-1 within the packaging material, and a label that indicates that the FXR modulator or composition, or pharmaceutically acceptable derivative thereof, is used for modulating the activity of farnesoid X receptor for treatment of one or more symptoms of at least one disease state characterized by elevated expression of LOX-1.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

Standard physiological, pharmacological and biochemical procedures are available for testing agents to identify those that possess biological activities that modulate the activity of the farnesoid X receptor. Such assays include, for example, biochemical assays such as binding assays, fluorescence polarization assays, FRET based coactivator recruitment assays (see generally Glickman et al., J. Biomolecular Screening, 7 No. 1 3-10 (2002)), as well as cell based assays including the co-transfection assay, the use of LBD-Gal 4 chimeras, protein-protein interaction assays (see, Lehmann. et al., J. Biol Chem., 272(6) 3137-3140 (1997), and gene expression assays.

High throughput screening systems are commercially available (see, e.g., Zymark Corp., Hopkinton, Mass.; Air Technical Industries, Mentor, Ohio; Beckman Instruments Inc., Fullerton, Calif.; Precision Systems, Inc., Natick, Mass.) that enable these assays to be run in a high throughput mode. These systems typically automate entire procedures, including sample and reagent pipetting, liquid dispensing timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

Assays that do not require washing or liquid separation steps can be used for high throughput screening systems and include biochemical assays such as fluorescence polarization assays (see for example, Owicki, J., Biomol Screen 2000 October; 5(5):297) scintillation proximity assays (SPA) (see for example, Carpenter et al., Methods Mol Biol 2002; 190:31-49) and fluorescence resonance energy transfer energy transfer (FRET) or time resolved FRET based coactivator recruitment assays (Mukherjee et al., J Steroid Biochem Mol Biol 2002 July; 81(3):217-25; (Zhou et al., Mol Endocrinol. 1998 October; 12(10):1594-604). Generally such assays can be preformed using either the full length receptor, or isolated ligand binding domain (LBD). In the case of the farnesoid X receptor, the LBD comprises amino acids 244 to 472 of the full length sequence.

If a fluorescently labeled ligand is available, fluorescence polarization assays provide a way of detecting binding of agents to the farnesoid X receptor by measuring changes in fluorescence polarization that occur as a result of the displacement of a trace amount of the label ligand by the agent. Additionally this approach can also be used to monitor the ligand dependent association of a fluorescently labeled coactivator peptide to the farnesoid X receptor to detect ligand binding to the farnesoid X receptor.

The ability of an agent to bind to a receptor, or heterodimer complex with RXR, can also be measured in a homogeneous assay format by assessing the degree to which the agent can compete off a radiolabelled ligand with known affinity for the receptor using a scintillation proximity assay (SPA). In this approach, the radioactivity emitted by a radiolabelled agent generates an optical signal when it is brought into close proximity to a scintillant such as a Ysi-copper containing bead, to which the farnesoid X receptor is bound. If the radiolabelled agent is displaced from the farnesoid X receptor the amount of light emitted from the farnesoid X receptor bound scintillant decreases, and this can be readily detected using standard microplate liquid scintillation plate readers such as, for example, a Wallac MicroBeta reader.

The heterodimerization of the farnesoid X receptor with RXRα can also be measured by fluorescence resonance energy transfer (FRET), or time resolved FRET, to monitor the ability of the agents provided herein to bind to the farnesoid X receptor or other nuclear receptors. Both approaches rely upon the fact that energy transfer from a donor molecule to an acceptor molecule only occurs when donor and acceptor are in close proximity. Typically the purified LBD of the farnesoid X receptor is labeled with biotin then mixed with stoichiometric amounts of europium labeled streptavidin (Wallac Inc.), and the purified LBD of RXRα is labeled with a suitable fluorophore such as CY5™. Equimolar amounts of each modified LBD are mixed together and allowed to equilibrate for at least 1 hour prior to addition to either variable or constant concentrations of the sample for which the affinity is to be determined. After equilibration, the time-resolved fluorescent signal is quantitated using a fluorescent plate reader. The affinity of the agent can then be estimated from a plot of fluorescence versus concentration of agent added.

This approach can also be exploited to measure the ligand dependent interaction of a co-activator peptide with a farnesoid X receptor in order to characterize the agonist or antagonist activity of the agents disclosed herein. Typically the assay in this case involves the use a recombinant Glutathione-5-transferase (GST)-farnesoid X receptor ligand binding domain (LBD) fusion protein and a synthetic biotinylated peptide sequenced derived from the receptor interacting domain of a co-activator peptide such as the steroid receptor coactivator 1 (SRC-1). Typically GST-LBD is labeled with a europium chelate (donor) via a europium-tagged anti-GST antibody, and the coactivator peptide is labeled with allophycocyanin via a streptavidin-biotin linkage.

In the presence of an agonist for the farnesoid X receptor, the peptide is recruited to the GST-LBD bringing europium and allophycocyanin into close proximity to enable energy transfer from the europium chelate to the allophycocyanin. Upon excitation of the complex with light at 340 nm excitation energy absorbed by the europium chelate is transmitted to the allophycocyanin moiety resulting in emission at 665 nm. If the europium chelate is not brought in to close proximity to the allophycocyanin moiety there is little or no energy transfer and excitation of the europium chelate results in emission at 615 nm. Thus the intensity of light emitted at 665 nm gives an indication of the strength of the protein-protein interaction. The activity of a farnesoid X receptor antagonist can be measured by determining the ability of a agent to competitively inhibit (i.e., IC₅₀) the activity of an agonist for the farnesoid X receptor.

DNA binding assays can be used to evaluate the ability of an agent to modulate farnesoid X receptor activity. These assays measure the ability of nuclear receptor proteins, including farnesoid X receptor and RXR, to bind to regulatory elements of genes known to be modulated by farnesoid X receptor. In general, the assay involves combining a DNA sequence which can interact with the farnesoid X receptors, and the farnesoid X receptor proteins under conditions, such that the amount of binding of the farnesoid X receptor proteins in the presence or absence of the agent can be measured. In the presence of an agonist, farnesoid X receptor heterodimerizes with RXR and the complex binds to the regulatory element. Methods including, but not limited to DNAse footprinting, gel shift assays, and chromatin immunoprecipitation can be used to measure the amount of farnesoid X receptor proteins bound to regulatory elements.

In addition a variety of cell based assay methodologies may be successfully used in screening assays to identify and profile the specificity of agents described herein. These approaches include the co-transfection assay, translocation assays, and gene expression assays.

Three basic variants of the co-transfection assay strategy exist, co-transfection assays using full-length farnesoid X receptor, co-transfection assays using chimeric farnesoid X receptors comprising the ligand binding domain of the farnesoid X receptor fused to a heterologous DNA binding domain, and assays based around the use of the mammalian two hybrid assay system.

The basic co-transfection assay is based on the co-transfection into the cell of an expression plasmid to express the farnesoid X receptor in the cell with a reporter plasmid comprising a reporter gene whose expression is under the control of DNA sequence that is capable of interacting with that nuclear receptor. (See for example U.S. Pat. Nos. 5,071,773; 5,298,429, 6,416,957, WO 00/76523). Treatment of the transfected cells with an agonist for the farnesoid X receptor increases the transcriptional activity of that receptor which is reflected by an increase in expression of the reporter gene, which may be measured by a variety of standard procedures.

For those receptors that function as heterodimers with RXR, such as the farnesoid X receptor, the co-transfection assay typically includes the use of expression plasmids for both the farnesoid X receptor and RXR. Typical co-transfection assays require access to the full-length farnesoid X receptor and suitable response elements that provide sufficient screening sensitivity and specificity to the farnesoid X receptor.

Genes encoding the following full-length previously described proteins, which are suitable for use in the co-transfection studies and profiling the agents described herein, include rat farnesoid X receptor (GenBank Accession No. NM_{—}021745), human farnesoid X receptor (GenBank Accession No. NM_{—}005123), human RXR α (GenBank Accession No. NM_{—}002957), human RXR β (GenBank Accession No. XM_{—}042579), human RXR γ (GenBank Accession No. XM_{—}053680),

Reporter plasmids may be constructed using standard molecular biological techniques by placing cDNA encoding for the reporter gene downstream from a suitable minimal promoter. For example luciferase reporter plasmids may be constructed by placing cDNA encoding firefly luciferase immediately down stream from the herpes virus thymidine kinase promoter (located at nucleotides residues −105 to +51 of the thymidine kinase nucleotide sequence) which is linked in turn to the various response elements.

Numerous methods of co-transfecting the expression and reporter plasmids are known to those of skill in the art and may be used for the co-transfection assay to introduce the plasmids into a suitable cell type. Typically such a cell will not endogenously express farnesoid X receptors that interact with the response elements used in the reporter plasmid.

Numerous reporter gene systems are known in the art and include, for example, alkaline phosphatase Berger, J., et al (1988) Gene 66 1-10; Kain, S. R. (1997) Methods. Mol. Biol. 63 49-60), β-galactosidase (See, U.S. Pat. No. 5,070,012, issued Dec. 3, 1991 to Nolan et al., and Bronstein, I., et al., (1989) J. Chemilum. Biolum. 4 99-111), chloramphenicol acetyltransferase (See Gorman et al., Mol Cell Biol. (1982) 2 1044-51), β-glucuronidase, peroxidase, β-lactamase (U.S. Pat. Nos. 5,741,657 and 5,955,604), catalytic antibodies, luciferases (U.S. Pat. Nos. 5,221,623; 5,683,888; 5,674,713; 5,650,289; 5,843,746) and naturally fluorescent proteins (Tsien, R. Y. (1998) Annu. Rev. Biochem. 67 509-44).

The use of chimeras comprising the ligand binding domain (LBD) of the farnesoid X receptor to a heterologous DNA binding domain (DBD) expands the versatility of cell based assays by directing activation of the farnesoid X receptor in question to defined DNA binding elements recognized by defined DNA binding domain (see WO95/18380). This assay expands the utility of cell based co-transfection assays in cases where the biological response or screening window using the native DNA binding domain is not satisfactory.

In general the methodology is similar to that used with the basic co-transfection assay, except that a chimeric construct is used in place of the full-length farnesoid X receptor. As with the full-length farnesoid X receptor, treatment of the transfected cells with an agonist for the farnesoid X receptor LBD increases the transcriptional activity of the heterologous DNA binding domain which is reflected by an increase in expression of the reporter gene as described above. Typically for such chimeric constructs, the DNA binding domains from defined farnesoid X receptors, or from yeast or bacterially derived transcriptional regulators such as members of the GAL 4 and Lex A/Umud super families are used.

A third cell based assay of utility for screening agents is a mammalian two-hybrid assay that measures the ability of the nuclear hormone receptor to interact with a cofactor in the presence of a ligand. (See for example, U.S. Pat. Nos. 5,667,973, 5,283,173 and 5,468,614). The basic approach is to create three plasmid constructs that enable the interaction of the farnesoid X receptor with the interacting protein to be coupled to a transcriptional readout within a living cell. The first construct is an expression plasmid for expressing a fusion protein comprising the interacting protein, or a portion of that protein containing the interacting domain, fused to a GAL4 DNA binding domain. The second expression plasmid comprises DNA encoding the farnesoid X receptor fused to a strong transcription activation domain such as VP16, and the third construct comprises the reporter plasmid comprising a reporter gene with a minimal promoter and GAL4 upstream activating sequences.

Once all three plasmids are introduced into a cell, the GAL4 DNA binding domain encoded in the first construct allows for specific binding of the fusion protein to GAL4 sites upstream of a minimal promoter. However because the GAL4 DNA binding domain typically has no strong transcriptional activation properties in isolation, expression of the reporter gene occurs only at a low level. In the presence of a ligand, the farnesoid X receptor-VP16 fusion protein can bind to the GAL4-interacting protein fusion protein bringing the strong transcriptional activator VP16 in close proximity to the GAL4 binding sites and minimal promoter region of the reporter gene. This interaction significantly enhances the transcription of the reporter gene, which can be measured for various reporter genes as described above. Transcription of the reporter gene is thus driven by the interaction of the interacting protein and farnesoid X receptor in a ligand dependent fashion.

An agent can be tested for the ability to induce nuclear localization of a nuclear protein receptor, such as farnesoid X receptor. Upon binding of an agonist, farnesoid X receptor translocates from the cytoplasm to the nucleus. Microscopic techniques can be used to visualize and quantitate the amount of farnesoid X receptor located in the nucleus. In some embodiments, this assay can utilize a chimeric farnesoid X receptor fused to a fluorescent protein.

An agent can also be evaluated for its ability to increase or decrease the expression of genes known to be modulated by the farnesoid X receptor in vivo, using Northern-blot, RT PCR or oligonucleotide microarray analysis to analyze RNA levels. Western-blot analysis can be used to measure expression of proteins encoded by farnesoid X receptor target genes. Additional genes known to be regulated by the farnesoid X receptor include cholesterol 7 α-hydroxylase (CYP7A1), the rate limiting enzyme in the conversion of cholesterol to bile acids, the small heterodimer partner (SHP), the bile salt export pump (BSEP, ABCB11), canalicular bile acid export protein, sodium taurocholate cotransporting polypeptide (NTCP, SLC10A1) and intestinal bile acid binding protein (I-BABP).

The DDAH1 gene is known to be induced by FXR. The inventors have demonstrated that ASS1 and GTPCH are also induced by FXR. Accordingly, in some embodiments of the methods of reducing expression of LOX-1, expression of at least one FXR target selected from DDAH1, ASS1, and GTPCH is increased. Those genes are involved in increasing nitric oxide production (and simultaneously decreasing ADMA). Thus, in some embodiments of the methods of reducing expression of LOX-1, ADMA levels are reduced. In some embodiments of the methods of reducing expression of LOX-1, nitric oxide synthase expression is increased.

Expression of a farnesoid X receptor target gene can be conveniently normalized to an internal control and the data plotted as fold induction relative to untreated or vehicle treated cells. A control agent, such as an agonist, may be included along with DMSO as high and low controls respectively for normalization of the assay data.

Any agent which is a candidate for modulation of the farnesoid X receptor may be tested by the methods described above. Generally, though not necessarily, agents are tested at several different concentrations and administered one or more times to optimize the chances that activation of the receptor will be detected and recognized if present. Typically assays are performed in triplicate, for example, and vary within experimental error by less than about 15%. Each experiment is typically repeated about three or more times with similar results.

In some embodiments, the effects of agents and compositions on farnesoid X receptor gene expression can be evaluated in animals. After the administration of agents, various tissues can be harvested to determine the effect of agents on activities directly or indirectly regulated by farnesoid X receptor.

Provided herein are methods involving both in vitro and in vivo uses of an agent that modulates farnesoid X receptor activity. Such agents will typically exhibit farnesoid X receptor agonist, partial agonist, partial antagonist or antagonist activity in one of the in vitro assays described herein.

Methods of altering farnesoid X receptor activity, by contacting the receptor with at least one agent, are provided.

Provided are methods of treating at least one disease state characterized by elevated expression of LOX-1 in a patient. Elevated expression of LOX-1 may be determined, for example, by measuring LOX-1 mRNA expression levels or by measuring LOX-1 protein levels, including for example by measuring the level of serum soluble LOX-1. A disease state is characterized by elevated expression of LOX-1 in a patient if patients with the disease exhibit a mean expression level of LOX-1 that is above the expression level of LOX-1 in a patients without the disease, and if that increased expression level is statistically significant.

Treatment with a farnesoid X receptor agonist may be associated with side effects. Provided herein is method of treating a disease state characterized by elevated expression of LOX-1 with an agent selected to have fewer side effects based on its profile and activities in assays testing for farnesoid X receptor activity. For example, a agent may be selected for high activity in treating features of the disease state characterized by elevated expression of LOX-1 and low activity in assays that do not monitor features of the disease state characterized by elevated expression of LOX-1.

Provided is a method for diagnosing the risk that a patient will develop at least one disease state characterized by elevated expression of LOX-1. This method comprises measuring the level or expression of FXR and/or the level of FXR activity in at least one tissue. Methods of measuring FXR expression include Northern-blot, RT PCR or oligonucleotide microarray analysis to analyze RNA levels and Western blot to measure protein levels. Methods of measuring FXR activity are described above.

Administering at least one farnesoid X receptor agonist can potentiate the effects of known agents useful for the treatment of the disease state characterized by elevated expression of LOX-1. Contemplated herein is combination therapy using at least one farnesoid X receptor agonist or a pharmaceutically acceptable derivative thereof, in combination with one or more of the following: cholesterol lowering agents (such as statins and ezitimibe), anti-inflammatory agents, and any prescribed drug for the targeted indication.

The farnesoid X receptor agonist, or pharmaceutically acceptable derivative thereof, is administered simultaneously with, prior to, or after administration of one or more of the above agents.

The invention is further illustrated by the following non-limiting examples.

### EXAMPLES

The cut off for statistical significance in the context of the following examples was p<0.05 unless otherwise specified.

### Example 1

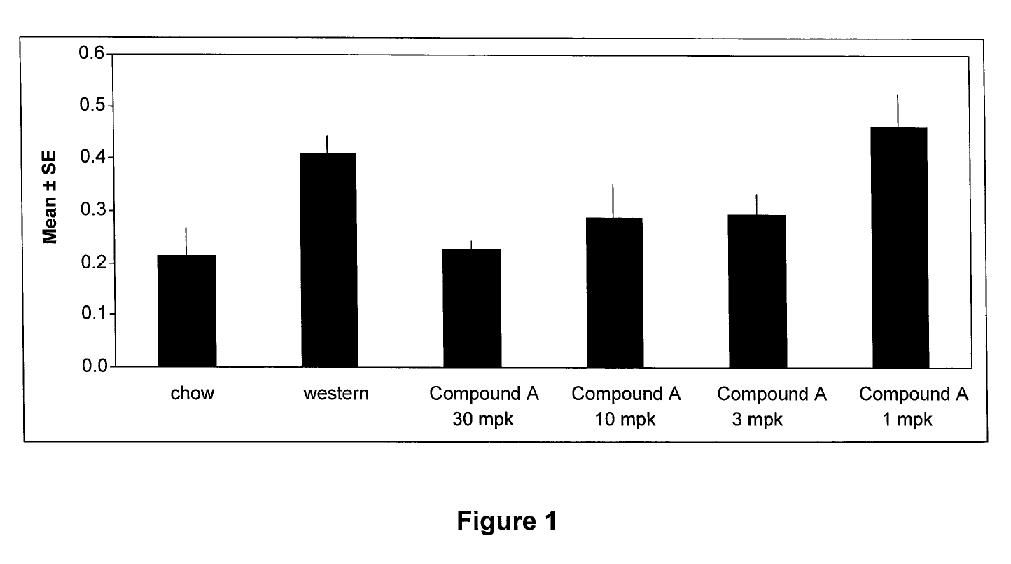

Since LOX-1 is regulated by oxLDL, LOX-1 mRNA expression was monitored in liver from LDLR−/− (LDLRKO) mice fed a western diet for 7 days. As shown in FIG. 1, hepatic expression of LOX-1 was induced 2-fold by the western diet compared to chow fed controls. Since FXR agonists have been shown to down regulate serum cholesterol and LDL levels in these mice, and by inference oxLDL, the effect of the FXR agonist, Compound A (isopropyl 3-(3,4-difluorobenzoyl)-1,1-dimethyl-1,2,3,6-tetrahydroazepino[4,5-b]indole-5-carboxylate), on LOX-1 mRNA expression was determined. LOX-1 mRNA induction by the western diet was dose dependently inhibited with increasing concentrations of Compound A (FIG. 1).

### Example 2

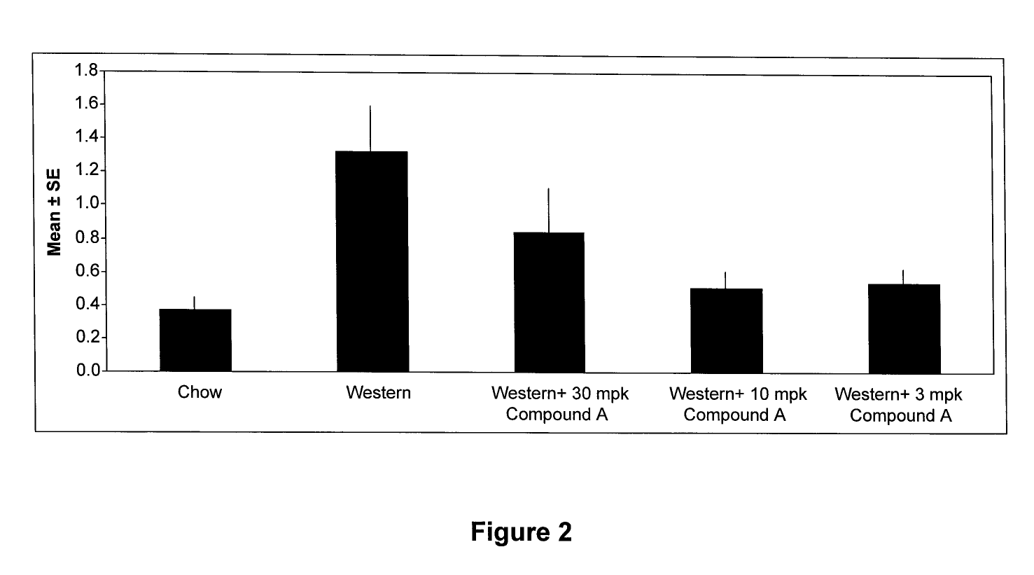

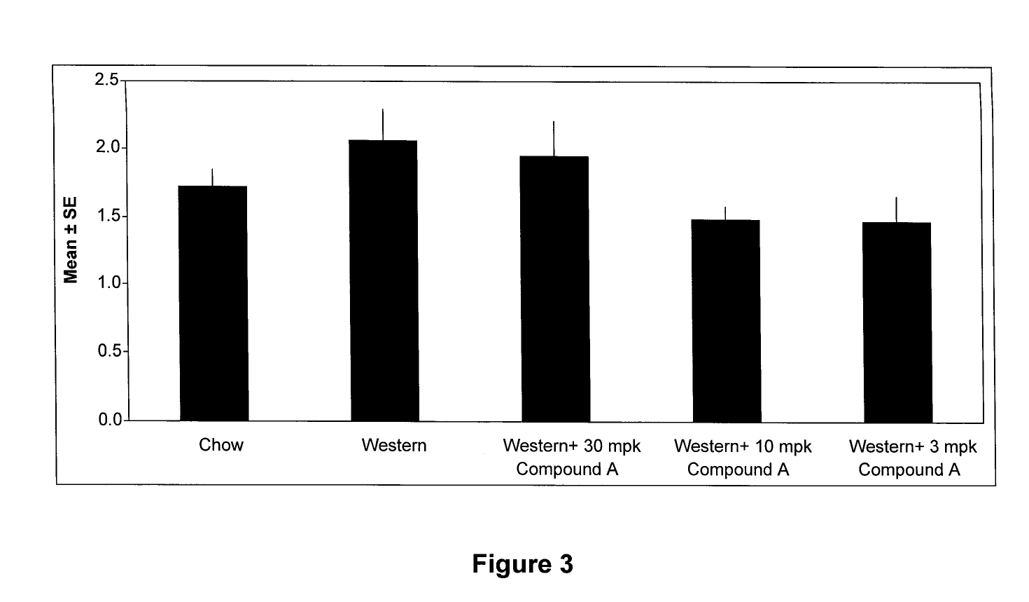

In addition, known LOX-1 target genes, such as VCAM-1, were significantly inhibited by compound A as well (FIG. 2). Importantly, the regulation of LOX-1 by FXR was specific, since expression of another oxLDL hepatic scavenger receptor, CD36, was not affected by compound A (FIG. 3).

### Example 3

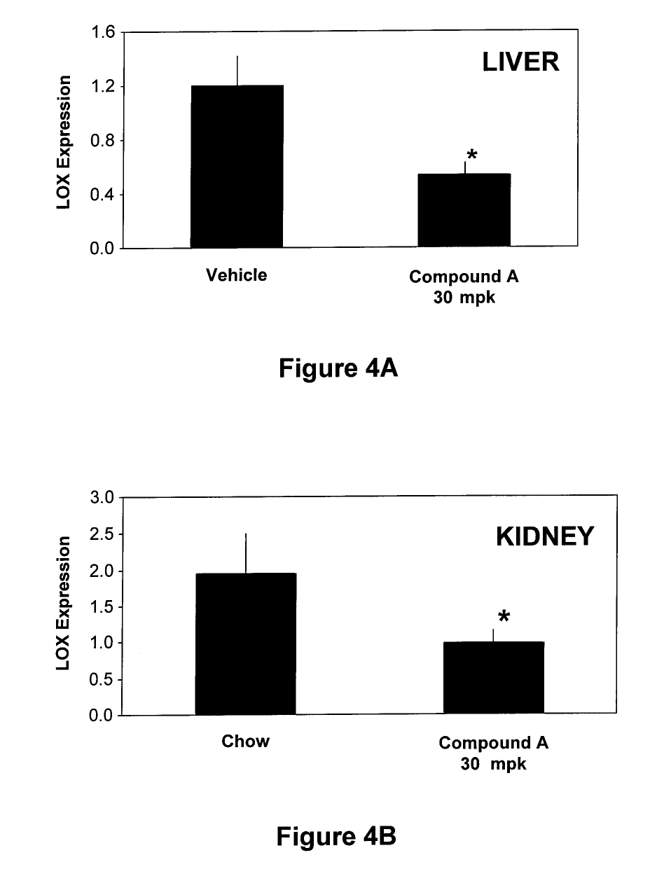

To determine whether FXR agonists could block LOX-1 gene expression under other settings, the regulation of LOX-1 by FXR was studied in the diabetic mouse strain, KKAy. As shown in FIG. 4A, treatment of KKAy mice on a chow diet with compound A for 7 days resulted in a significant repression of hepatic LOX-1 expression. The renal regulation of LOX-1 was also determined since FXR is also expressed in the kidney. As shown in FIG. 4B, renal LOX-1 expression was also significantly inhibited by compound A.

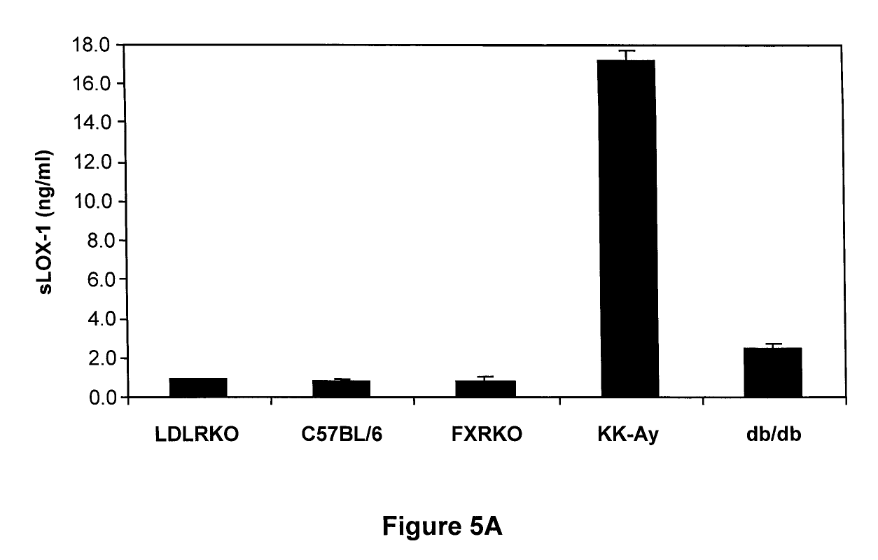

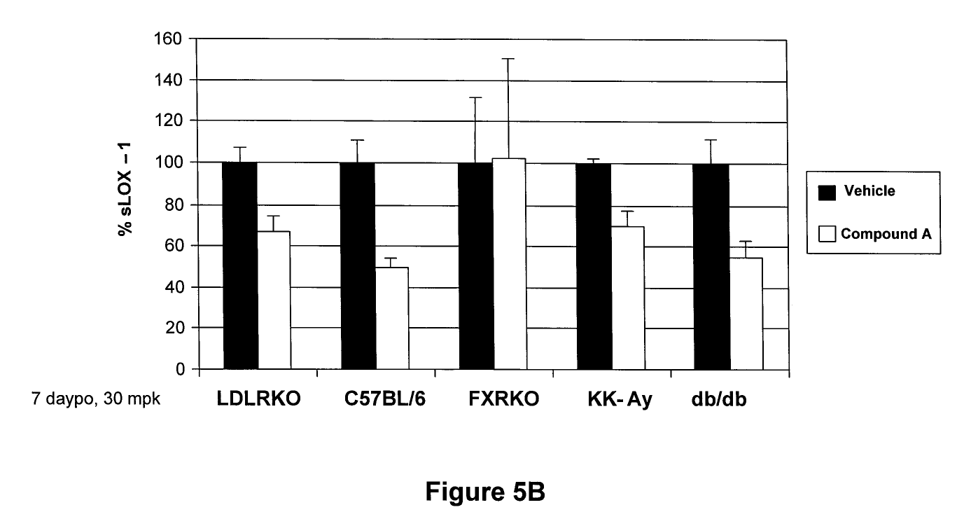

Moreover, the expression of soluble LOX-1 (sLOX-1) in the serum was also monitored using an ELISA based assay after Compound A treatment. As shown in FIG. 5A, serum sLOX levels are elevated in KKAy mice compared to other strains. Compound A treatment significantly reduced sLOX-1 in various mouse strains fed a high fat diet and treated with 30 mpk of Compound A orally and daily for 7 days. (FIG. 5B). The results for the FXR KO mouse strain presented in FIG. 5B also show that sLOX-1 reduction is dependent upon FXR.

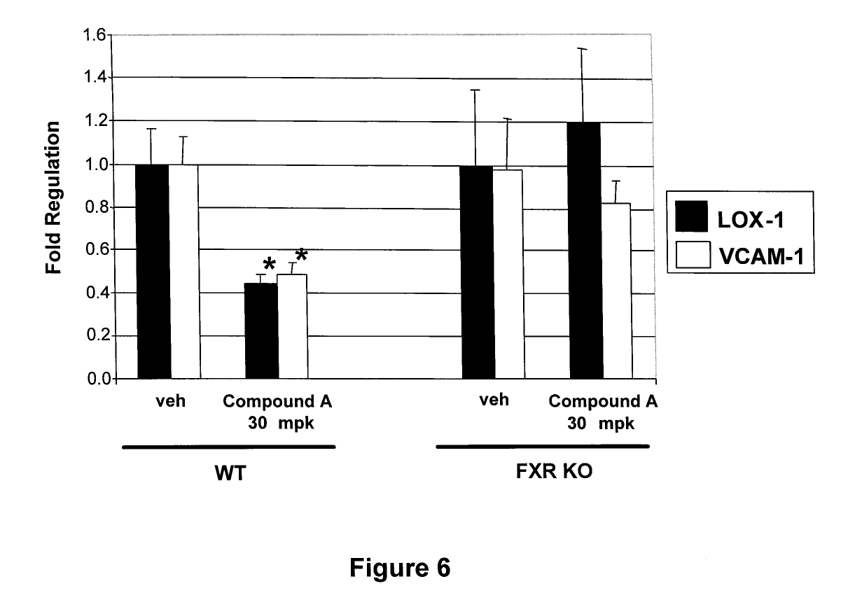

Furthermore, inhibition of LOX-1 and VCAM-1 gene expression is dependent upon FXR (FIG. 6; * p<0.01 vs. the chow vehicle control.). Male FXR deficient (FXR KO) or wildtype mice on a chow diet supplemented with 0.5% chenodeoxycholic acid (CA) were treated orally daily with 30 mpk of Compound A for 7 days, and hepatic gene expression of LOX-1 and VCAM-1 was determined by real-time PCR.

These data demonstrate that FXR can antagonize LOX-1 expression at both the transcriptional and protein level.

While some embodiments have been shown and described, various modifications and substitutions may be made thereto without departing from the spirit and scope of the invention. For example, for claim construction purposes, it is not the literal language thereof, and it is thus not intended that exemplary embodiments from the specification be read into the claims. Accordingly, it is to be understood that the present invention has been described by way of illustration and not limitations on the scope of the claims.

1. A method of treating at least one disease state characterized by elevated expression of the Lectin-like Oxidized Low-density Lipoprotein Receptor 1 (LOX-1) in a patient, the method comprising administering to the patient a therapeutically effective amount of at least one farnesoid X receptor (FXR) agonist, wherein the at least one FXR agonist reduces expression of LOX-1 in the patient.

2. The method of claim 1, wherein the disease state is further characterized by at least one of endothelial dysfunction and vascular inflammation.

3. The method of claim 1, wherein the at least one disease state is selected from heart failure, myocardial injury, atherosclerosis, diabetic nephropathy, hypertension, sepsis, osteoarthritis, and rheumatoid arthritis.

4. The method of claim 3, wherein the heart failure comprises at least one of left sided heart failure, right sided heart failure, systolic heart failure, and diastolic heart failure.

5. The method of claim 3, wherein the myocardial injury comprises at least one of unstable angina and myocardial infarction.

6. The method of claim 1, wherein the FXR agonist reduces at least one of NF-κB pathway signaling, MAPK pathway signaling, and production of reactive oxygen species in the patient.

7. The method of claim 1, wherein the FXR agonist increases nitric oxide production in the patient.

8. The method of claim 1, wherein LOX-1 expression is reduced in at least one tissue of the patient selected from heart, liver, and kidney.

9. The method of claim 1, wherein LOX-1 expression is reduced in at least one cell type of the patient selected from endothelial cells, macrophages, smooth muscle cells, dendritic cells, cardiac myocytes, and platelets.

10. The method of claim 1, wherein the level of serum soluble LOX-1 protein in the patient is reduced.

11. The method of claim 1, wherein expression of at least one LOX-1 target selected from MCP-1, VCAM-1, and ICAM-1 is reduced in the patient.

12. The method of claim 1, wherein expression of at least one FXR target selected from DDAH1, ASS1, and GTPCH is increased in the patient.

13. The method of claim 1, wherein the level of assymetric dimethylarginine (ADMA) is reduced in the patient.

14. The method of claim 13, wherein expression of nitric oxide synthase is increased in the patient.

15. The method of claim 1, wherein the LOX-1 expression level in the patient is reduced to about the level of LOX-1 expression in the absence of the disease state.

16. The method of claim 1, wherein the LOX-1 expression level in the patient is reduced to below about a threshold level of LOX-1 expression.

17. The method of claim 16, wherein the threshold level of LOX-1 expression is higher than the level of LOX-1 expression in the absence of the disease state.

18. The method of claim 1, wherein the at least one FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl) 1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoyl)-1,4,4-trimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro-4H-thieno[2,3-D]azepine-8-carboxylic acid ethyl ester;
6-(4-fluoro-benzoyl)-3,6,7,8-tetrahydro-imidazo[4,5-D]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide;
diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate;
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3(4-fluorobenzoyl)-2-methyl-1,2,3,6-tetrahydroazepino[4,5-b]indole-5-carboxylate; and
n-propyl 3(4-fluorobenzoyl)-2-methyl-8-fluoro-1,2,3,6-tetrahydroazepino[4,5-b]indole-5-carboxylate.

19. A method of reducing expression of LOX-1 in a cell, comprising administering an effective amount of at least one FXR agonist, to thereby reduce expression of LOX-1 in the cell.

20. The method of claim 19, wherein the FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl) 1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoyl)-1,4,4-trimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro-4H-thieno[2,3-D]azepine-8-carboxylic acid ethyl ester;
6-(4-fluoro-benzoyl)-3,6,7,8-tetrahydro-imidazo[4,5-D]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide;
diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate;
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3(4-fluorobenzoyl)-2-methyl-1,2,3,6-tetrahydroazepino[4,5-b]indole-5-carboxylate; and
n-propyl 3(4-fluorobenzoyl)-2-methyl-8-fluoro-1,2,3,6-tetrahydroazepino[4,5-b]indole-5-carboxylate.

21. The method of claim 19, wherein the FXR agonist reduces at least one of NF-κB pathway signaling, MAPK pathway signaling, and production of reactive oxygen species by the cell.

22. The method of claim 19, wherein the FXR agonist increases nitric oxide production by the cell.

23. The method of claim 19, wherein expression of at least one LOX-1 target selected from MCP-1, VCAM-1 and ICAM-1 is reduced in the patient.

24. The method of claim 19, wherein expression of at least one FXR target selected from DDAH1, ASS1, and GTPCH is increased in the patient.

25. The method of claim 19, wherein the level of ADMA is reduced in the patient.

26. The method of claim 25, wherein expression of nitric oxide synthase is increased in the patient.
